(19) United States
(12) Patent Application Publication
(10) Pub. No.: US 2007/0287661 A1
(43) Pub. Date: Dec. 13, 2007

(54) COMPOSITIONS AND METHODS FOR PREVENTION OR TREATMENT OF THROMBOCYTOPENIA AND METHODS FOR MANUFACTURING SAID COMPOSITIONS

(76) Inventor: Jianjun LU, Chengdu (CN)

Correspondence Address: MATTHIAS SCHOLL, 14781 MEMORIAL DRIVE, SUITE 1319, HOUSTON, TX, 77079, US

(21) Appl. No.: 11/762,262

(22) Filed: Jun. 13, 2007

(30) Foreign Application Priority Data
Jun. 13, 2006 (CN) 200610021167.1

(51) Int. Cl. A61K 38/39

(52) U.S. Cl. 514/2; 530/357

(57) ABSTRACT

This invention teaches a composition of peptides, amino acids, and microelements derived from keratin-containing animal material for treating thrombocytopenia and a method for manufacturing thereof, comprising hydrolyzing keratin-containing animal materials, and particularly animal nails, to obtain a hydrolysate; filtering and concentrating the hydrolysate; admixing alcohol; and purifying the resultant solution. In addition, the invention teaches a method for treating (curing and/or preventing) thrombocytopenia by administering parenterally to a patient a pharmaceutical composition comprising an excipient and a composition of peptides, amino acids, and microelements derived from keratin-containing animal material.

### CROSS-REFERENCE TO RELATED APPLICATIONS

This application claims priority to Chinese Patent Application No. 200610021167.1 filed on Jun., 13, 2006, the contents of which are incorporated herein by reference.

### BACKGROUND OF THE INVENTION

1. Field of the Invention

This invention pertains to the field of pharmacology and, in particular, to compositions derived from keratin-containing animal material, methods for manufacturing the same, and methods for using the same to increase platelet count in individuals affected with thrombocytopenia or at risk of developing thrombocytopenia.

2. Description of the Related Art

Platelets are small anucleate disk-shaped cells derived by cytoplasmic fragmentation of the precursor stem cells, megakaryocytes, found in bone marrow. They play an important role in blood coagulation. After formation in the bone marrow, platelets travel through the spleen and into the blood. During this process approximately one third of the platelets becomes sequestered in the spleen. The platelets which are transported to the blood reach a normal concentration in the blood of between 150,000 and 400,000 per microliter and have an average lifetime of about eight days. They play a crucial role in hemostasis. When the level of platelets falls below normal, the risk of hemorrhage increases. The condition associated with low levels of platelets is referred to as thrombocytopenia.

Ordinarily when the level of circulating platelets decreases a feedback mechanism is initiated which results in increased production of platelets. However, many pathophysiological conditions can cause thrombocytopenia. These include: a decreased production of platelets in the bone marrow (reduced thrombopoiesis); an accelerated destruction of platelets; or an increased splenic sequestration of platelets.

A decreased production of bone marrow may result from myelosuppression (reduction in the ability of the bone marrow to produce blood cells) as a consequence of gamma irradiation, therapeutic exposure to radiation, or cytotoxic drug treatment. Chemicals, and especially aromatic hydrocarbons, such as benzene or anthracene, can cause myelosuppression, resulting in thrombocytopenia. Additionally, rare bone marrow disorders such as congenital megakaryocytic hypoplasia and thrombocytopenia with absent radii (TAR syndrome) can selectively decrease megakaryocyte production, resulting in thrombocytopenia.

Thrombocytopenia resulting from accelerated destruction is caused by either an immunologic disorder or a non-immunologic disorder. Immunologic thrombocytopenia is caused, for example, by autoimmune disorders such as idiopathic thrombocytopenic purpura (ITP), viral or bacterial infections, and drugs. Non-immunologic thrombocytopenia is caused by vasculitis, hemolytic uremic syndrome, thrombotic thrombocytopenic purpura (ITP), disseminated intravascular coagulation (DIC) and prosthetic cardiac valves.

Increased splenic sequestration of platelets is typically caused by portal hypertension secondary to liver disease, splenic infiltration with tumor cells in myeloproliferative or lymphoproliferative disorders, or macrophage storage disorders such as Gaucher's disease.

Conventionally, in order to successfully treat thrombocytopenia, the cause for the decrease in platelet levels must be identified and then a drug or a procedure which will eliminate the underlying cause of thrombocytopenia must be administered. However, many causes of thrombocytopenia, such as, e.g., idiopathic thrombocytopenia purpura (ITP), are difficult or impossible to cure.

Accordingly, there urgently exists a need for therapeutic methods for increasing platelet levels or preventing significant decreases in platelet levels in patients in need of such increase or prevention. Ideally, the therapy should cure thrombocytopenia in patients exhibiting thrombocytopenia or prevent thrombocytopenia in patients at risk of developing thrombocytopenia. Such therapy preferably should be easy and safe to administer and should be effective for all types of thrombocytopenias regardless of the underlying pathophysiological conditions causing reduced platelet levels.

### SUMMARY OF THE INVENTION

In view of the above-described need, it is one objective of the invention to provide a composition of peptides, amino acids, and microelements derived from keratin-containing animal material suitable for parenteral administration for treating (curing, reducing, alleviating the symptoms of, and/or preventing) thrombocytopenia, the composition having such features as good water solubility, excellent bioavailability, limited molecular weight, high efficacy and excellent safety.

To achieve the above objectives, in accordance with one aspect of the present invention, there is provided a method of producing a composition of peptides, amino acids, and microelements derived from keratin-containing animal material for treating thrombocytopenia, comprising: (a) hydrolyzing keratin-containing animal material to obtain a hydrolysate; (b) filtering and concentrating said hydrolysate obtained in (a); (c) admixing alcohol; and (d) purifying.

In certain preferred embodiments, the keratin-containing animal material is animal nails. In certain most preferred embodiments, the animal nails are nails of pig, cattle and/or sheep. The term “nail” as used herein refers to a horn-like piece at the end of an animal's finger and/or toe. The term nails as used herein also encompasses animal claws.

In certain embodiments of the invention, the hydrolysis of keratin-containing animal materials is performed under acidic, alkaline, or enzymatic conditions (with a protease); and particularly, under acidic conditions in aqueous hydrochloric acid.

In certain embodiments of the invention, the hydrolysis of keratin-containing animal materials is performed at a temperature of between 30 and 150° C., particularly at between 120 and 150° C., and more particularly at 138° C.

In certain embodiments, the alcohol used in the methods of the invention is ethanol, and particularly 70% (140 proof) ethanol.

In certain embodiments of the invention, after the alcohol is admixed, the resultant solution is purified by (i) concentrating the solution; (ii) adjusting the pH to between about 6.5 and 7.5, and/or between about 8 and 10, and/or between about 2 and 4; (iii) sedimenting; and (iv) filtering through at most 2.0 μm fibrous membrane. Additionally, the filtrate is further filtered through at most an 80 kDa molecular size membrane.

In other aspects, the invention provides a composition of peptides, amino acids, and microelements derived from keratin-containing animal material for treating thrombocytopenia comprising peptides, amino acids (aspartate, serine, arginine, glycin, threonine, proline, alanine, valine, methionine, isoleucine, leucine and phenylalanine) as well as microelements including calcium, zinc and iron.

In certain embodiments of the invention, the composition of peptides, amino acids, and microelements derived from keratin-containing animal material for treating thrombocytopenia is prepared by any of the methods described herein.

In certain embodiments of the invention, the molecular weight of proteins present in the composition of peptides, amino acids, and microelements derived from keratin-containing animal material for treating thrombocytopenia is not higher than about 80 kDa as a result of cleaving and purification through ultrafiltration.

In other aspects, the invention provides a method of treating thrombocytopenia in a patient in need of such treatment (incl. prevention, alleviation of symptoms of, and curing) comprising administering to the patient a pharmaceutical composition prepared by any of the methods described herein, and/or comprising peptides, amino acids (aspartate, serine, arginine, glycin, threonine, proline, alanine, valine, methionine, isoleucine, leucine and phenylalanine) as well as microelements including calcium, zinc and iron.

In certain embodiments of the invention, the method of treating thrombocytopenia comprises administering a pharmaceutical composition comprising a suitable pharmaceutical excipient and a composition of peptides, amino acids, and microelements derived from keratin-containing animal material parenterally, and particularly, intravenously and/or intramuscularly.

### DESCRIPTION OF THE INVENTION

A method is provided for treating thrombocytopenia in a subject exhibiting thrombocytopenia, or at risk of developing thrombocytopenia. As used herein, “thrombocytopenia” is a disorder in which the platelet levels in the affected individual fall below a normal range of platelets for that individual.

The term “thrombocytopenia” as used herein includes infection-induced thrombocytopenia, treatment-induced thrombocytopenia, and physiologically-induced thrombocytopenia. Infection-induced thrombocytopenia is a disorder characterized by a low level of platelets in peripheral blood which is caused by an infectious agent such as a bacteria or viruses. Treatment-induced thrombocytopenia is a disorder characterized by a low level of platelets in peripheral blood which is caused by therapeutic treatments such as gamma irradiation, therapeutic exposure to radiation, cytotoxic drugs, and other chemicals, e.g., aromatic hydrocarbons. Physiologically-induced thrombocytopenia is a disorder characterized by a low level of platelets in peripheral blood which is caused by any mechanism other than infectious agents or therapeutic treatments causing thrombocytopenia.

As used herein, “a subject at risk of developing thrombocytopenia” is a subject who has a higher than normal probability of acquiring or developing thrombocytopenia. For example, a patient with a malignant tumor who is prescribed a chemotherapeutic treatment is at risk of developing treatment-induced thrombocytopenia and a subject who has an increased risk of exposure to infectious agents is at higher than normal risk of developing infection-induced thrombocytopenia.

One embodiment of the present invention is a method of treating thrombocytopenia. The method comprises the step of administering to a subject exhibiting thrombocytopenia a composition of peptides, amino acids, and microelements derived from keratin-containing animal material that stimulates, when administered parenterally in vivo, an increase in platelet counts in a thrombocytopenic mammal.

The composition of peptides, amino acids, and microelements derived from keratin-containing animal material is administered in an amount effective to increase platelet counts in the subject. An amount effective to increase platelet counts in the subject is an amount which causes an increase in the count of circulating platelet levels. The actual levels of platelets achieved will vary depending on many variables such as the initial status of the immune system in the subject, i.e., whether the subject has mild to severe thrombocytopenia (e.g., resulting from an autoimmune disease or splenic sequestration). In general, the platelet levels of a subject who has severe thrombocytopenia will initially be very low. Any increase in the platelet levels of such a subject, even increases to a level which are still below a normal level, can be advantageous to the subject.

The platelet levels of a subject at risk of developing thrombocytopenia, on the other hand, are generally within a normal range. The composition of peptides, amino acids, and microelements derived from keratin-containing animal material prevents the platelet levels of such a subject from decreasing to a level which would cause thrombocytopenia. Thus, administering the composition of peptides, amino acids, and microelements derived from keratin-containing animal material to the subject will inhibit to a medically significant extent, the decrease in platelet count that would otherwise occur in the absence of treatment according to the invention thereby preventing the development of thrombocytopenia. Preferably the effective amount is one which prevents platelet levels from decreasing below a level of 50,000 platelets per microliter.

An effective amount of a composition of peptides, amino acids, and microelements derived from keratin-containing animal material for increasing platelet levels may be measured by any conventional method known in the art for measuring platelet levels or for measuring parameters which correlate with platelet levels. Platelet count is determined simply by obtaining a blood sample and counting the number of platelets per microliter of blood. Platelet count also can correlate with bleeding time, a measure of platelet levels.

A composition of peptides, amino acids, and microelements derived from keratin-containing animal material for treating thrombocytopenia suitable for parenteral administration is prepared with the adoption of the following methods. Keratin-containing animal materials, and specifically, nails of pig, cattle and/or sheep, are cleaned with water and subjected to hydrolysis under acidic, alkaline or enzymatic conditions at a temperature between 30-150°.

Following hydrolysis, the liquid phase is collected, subjected to sedimentation in a cool place, and filtered. The filtrate is optionally concentrated, and alcohol (e.g., ethanol or propanol) is admixed. The mixture is subjected to sedimentation in a cool place, and filtered. The filtrate is optionally concentrated, the pH of the filtrate is adjusted to about 7, subjected to sedimentation in a cool place, and filtered.

Thereafter, pH is adjusted to between 6.5-7.5, e.g., with 20% NaOH aq., the filtrate subjected to sedimentation in a cool place and filtered using a fibrous membrane with a pore size of less than 2.0 μm.

Thereafter, pH is adjusted to between 8 and 10, e.g., with 20% NaOH aq., and the filtrate is first subjected to steam heating for 5-30 minutes at the temperature range of 80-100° C. before being subjected to sedimentation in a cool place and filtered using a fibrous membrane with a pore size of less than 2.0 μm.

Thereafter, pH is adjusted to between 2 and 4, e.g., with 20% HCl aq., and the filtrate is first subjected to steam heating for 5-30 minutes at the temperature range of 80-100° C. before being subjected to sedimentation in a cool place and filtered using a fibrous membrane with a pore size of less than 2.0 μm.

The filtrate is then passed through a membrane with a molecular size of not higher than 80 kDa (kilodalton).

The resultant solution is optionally purified with active carbon, mixed with a pH regulator, and other excipients, and sterilized giving rise to a pharmaceutical composition comprising peptides, amino acids, and microelements derived from keratin-containing animal material, which is suitable for parenteral administration; and optionally, further evaporated to produce a lyophilized powder which made be later reconstituted to make a pharmaceutical composition suitable for parenteral administration.

The composition so prepared comprises peptides, amino acids (aspartate, serine, arginine, glycin, threonine, proline, alanine, valine, methionine, isoleucine, leucine and phenylalanine) as well as such microelements as calcium, zinc and iron. It enhances the metabolism and immunity of the body, and promotes proliferation, differentiation, maturation and release of hemocytes. It significantly boosts up the levels of leucocytes and platelets.

The a composition of peptides, amino acids, and microelements derived from keratin-containing animal material effectively improves hemostasis, maturation of megakarycocytes, increases the number of leucocytes, and returns concentrations of platelets in blood to normal levels in subjects suffering from thrombocytopenia of various types. It enhances the normal defense functions of the body, has minimum acute and long term toxicity, and is safe for human administration. In addition, the composition of peptides, amino acids, and microelements derived from keratin-containing animal material has excellent bioavailability and excellent water solubility and can be administered parenterally, which overcomes problems with absorption by gastrointestinal biomembrane exhibited by conventional drugs used to treat thrombocytopenia.

This invention is not to be limited to the specific embodiments disclosed herein and modifications for various applications and other embodiments are intended to be included within the scope of the appended claims. While this invention has been described in connection with particular examples thereof, the true scope of the invention should not be so limited since other modifications will become apparent to the skilled practitioner upon a study of the drawings, specification, and following claims.

All publications and patent applications mentioned in this specification are indicative of the level of skill of those skilled in the art to which this invention pertains. All publications and patent applications mentioned in this specification are herein incorporated by reference to the same extent as if each individual publication or patent application mentioned in this specification was specifically and individually indicated to be incorporated by reference.

### EXAMPLES

Embodiments of the present invention provide clinical benefits to subjects exhibiting thrombocytopenia or at risk of developing thrombocytopenia. These benefits are highlighted in the Examples which follow.

### Example 1

### Preparation of a Composition of Peptides, Amino Acids, and Microelements Derived from Keratin-Containing Animal Material Suitable for Parenteral Administration

A. Pre-treating keratin-containing animal materials (pig nails): Pig nails were cleaned by rinsing with water, and then soaked over 12 hours in 10 times their volume of warm water at 40-60° C. The nails were dried. The nails were then subjected to 10 times their volume of warm 0.2-0.5% NaOH (w/v) solution at a temperature of between 40-60° C. for 1 hour. The nails were rinsed with water until the pH of discarded water was less than 7. Any skin attached to the nails was removed by cutting.

B. Hydrolyzing keratin-containing animal material (pig nails): Pig nails pretreated as above in A. were washed three times before being placed in a hydrolysis vessel. 20 times their volume of 0.1 M HCl aq. was added and hydrolysis was carried out at a temperature of about 138° C. for 3 hours. The liquid hydrolysate was poured off and hydrolysis of the remaining solid materials was repeated for additional 3 times with fresh HCl aq. solution. The hydrolysates were combined and were allowed to cool. Solid particles were removed by sedimentation at a temperature of between 2 to 10° C. The liquid phase was optionally stored at the same temperature.

C. Evaporating the hydrolysate: The liquid phase obtained as above in B. was filtered through a medium grade filter paper. The filtrate was transferred into an evaporator, and the volume of the filtrate was reduced by ⅓ by steam heating under reduced pressure of less than −0.05 MPa while maintaining the liquid surface boiling. The concentrate was allowed to cool to room temperature.

D. Precipitating with ethanol: Cold 70% ethanol at a temperature of 5° C. was added to the aqueous concentrate obtained as above in C. The liquids were mixed. A precipitate off was separated by sedimentation. The volume of the liquid phase was reduced by ⅓ by steam heating under reduced pressure of less than −0.05 MPa while maintaining the liquid surface boiling. The pH was adjusted to about 7 by adding an appropriate amount of 20% NaOH aq. The mixture was allowed to stand at a temperature of about 5° C. Any solids present were separated by sedimentation, followed by filtration through a membrane with a 1.0 μm pore size or a 2.0 μm pore size, and additionally, followed by filtration through 80 kDa molecular size separation filters (Millipore Corporation).

E. Further purifying and sterilizing: Filtrate obtained as above in D. containing 0.2-20 mg/mL of peptides was placed into a batching pan. 0.1-1.0% NaCl, 0.005-0.500% active carbon, and sterile water suitable for injection were added. The mixture was steam-heated to a temperature of 60-70° C. for 30 minutes. Active carbon was removed by filtration through a filter having a pore size of 0.8 μm. An appropriate amount of 10 M HCl or NaOH to adjust the pH value to 7.0 was added. Sterilization was carried out with 0.25 μm fibrin film or by any other suitable method.

F. Packaging and inspection: The solution obtained as above in E. was sealed into ampoules. The ampoules were steam-sterilized at a controlled temperature of 121° C. and pressure of 0.1 MPa for 45 minutes. Water was used to shower the ampoules after sterilization to test for leaks. The ampoules were then inspected to check for clarity.

### Example 2

### Preparation of Powder-Form Composition of Peptides, Amino Acids, and Microelements Derived from Keratin-Containing Animal Material Suitable for Reconstitution with an Excipient and Parenteral Administration

The procedure was carried out as in Example 1 from step A-D. Steps G. and H. were performed in place of steps G. and F. thereafter as described below.

G. Further purifying and sterilizing: Filtrate obtained as above in D. containing 2-50 mg/mL of peptides was placed into a batching pan. 0.1-1.0% NaCl aq, 0.005-0.500% active carbon, 0.2-10% mannitol, and sterile water suitable for injection were added. The mixture was steam-heated to a temperature of 60-70° C. for 30 minutes. Active carbon was removed by filtration through a filter having a pore size of 0.8 μm. An appropriate amount of 10 M HCl or NaOH to adjust the pH value to 7.0 was added. Sterilization was carried out with 0.25 μm fibrin film or by any other suitable method.

H. Drying and packaging: The solution obtained as above in G. was optionally stored until it was dried at reduced pressure of −0.01 kPa. The temperature during drying was kept below the eutectic point of 30° C. The obtained solid powder was distributed into ampoules and sealed. The ampoules were tested for leakage.

### Example 3

### Effect on Blood Coagulation Time

Plasma samples were obtained from healthy rabbits before intramuscular administration of the solution prepared as in Example 1 and repeatedly after onset of administration for measurement of the activated partial thromboplastin time (aPTT) according to Larrieu et al. (Rev. Hematol. 1957; 12: 199-210). The coagulation time after administration of three dosages was reduced by 9.07% (P>0.05), 34.06% (P<0.05), and 43.6% (P<0.01), respectively.

### Example 4

### Acute Toxicity

Stomachs of three groups of healthy mice with average weights of 40 g, 65 g, and 80 g were perfused with solutions made from dry powder prepared according to Example 2 for periods of 7 and 14 days. No mice have exhibited toxic symptoms or expired during that time. As compared with a control group, mice that were administered treatment exhibited small, but statistically-insignificant (P>0.05) weight increase.

### Example 5

### LD₅₀

LD₅₀ of mice subjected to intravenous injection of solution prepared as in Example 1 at various dosages was determined to be about 1.3 g/kg.

### Example 6

### Long-Term Toxicity

Groups of healthy mice with average weights of 100 g and 130 g were administered solution prepared as in Example 1 by intramuscular injection at low, intermediate, and high doses of 40 mg/kg, 60 mg/kg, and 80 mg/kg, respectively, over a period of 6 consecutive days, and were monitored for 3 months. The dosages corresponded to 80, 120, and 160 times the amount used for clinical application in humans. During the observation period, neither blood indices, incl. WBC, RBC, HGB, RC, PLT, HCT, MCV, MCt-I, MCtC, W-SCR, W-L, CR, W-SCC, W-LCC, RDW-CV, PDW and MPV, nor behavior, food consumption, or weight showed any signs of toxic symptoms.

With regard to blood biochemical indexes, liver and kidney functions have been tested, and showed no obvious discrepancy in AST, ALT, ALP, BU, TP, ALB, Glu, T-Bil, Crea and T-Cho between administration group and control group. By histopathological inspection, weight parameters for 13 organs of big mouse as represented by brain, heart, liver, spleen, lung, kidney, adrenal gland, thymus, womb, ovary, testis, epididymis and prostate were ascertained and were not found to show any obvious change and toxic influence. Furthermore, no pathological damage or change to structural features were observed in such organs as brain, spinal cord, heart, liver, spleen, lung, kidney, adrenal gland, thyroid gland, thymus, stomach, pancreas, ileum, bladder, womb, ovary, testis, epididymis and prostate of mice.

### Example 7

### Effect on Platelet Concentration

Blood samples were obtained from mice suffering from thrombocytopenia divided into groups with an average weight of 100 g and 130 g before intramuscular administration of the solution prepared as in Example 1 and repeatedly after onset of administration. Low, intermediate, and high doses of the solution were administered over a period of 3 months, on 6 consecutive days of each week only with a one day break in between. At the conclusion, groups that were administered high, intermediate, and low doses witnessed an increase in platelet concentration of 296.20% (P<0.01), 154.27% (P<0.05) and 18.36% (P>0.05), respectively, as compared with a control group. The gradual increase in platelet concentration during administration continued for a period of about 2 weeks after cessation of administration, and the high and intermediate dosages have witnessed an additional increase of 156.72% and 84.92% (P<0.05), respectively

### Example 7

### Effect on Megakarycocyte Concentration

Blood samples were obtained from mice suffering from thrombocytopenia divided into groups with an average weight of 100 g and 130 g before intramuscular administration of the solution prepared as in Example 1 and repeatedly after onset of administration. Low, intermediate, and high doses of the solution were administered over a period of 3 months, on 6 consecutive days of each week only with a one day break in between. At the conclusion, groups that were administered high, intermediate, and low doses witnessed an increase in megakarycocyte concentration by 36.04% (P<0.01), 27.30% (P<0.05) and 11.36% (P>0.05), respectively as compared with a control group.

### Example 8

### Effect on Leucocyte Concentration

Blood samples were obtained from mice suffering from thrombocytopenia divided into groups with an average weight of 100 g and 130 g before intramuscular administration of the solution prepared as in Example 1 and repeatedly after onset of administration. Low, intermediate, and high doses of the solution were administered over a period of 3 months, on 6 consecutive days of each week only with a one day break in between. At the conclusion, groups that were administered high, intermediate, and low doses witnessed an increase in leucocyte concentration by 28.00% (P<0.01), 21.09% (P<0.05) and 9.03% (P>0.05), respectively as compared with a control group.

What is claimed is:

1. A method for manufacturing a composition of peptides, amino acids, and microelements derived from keratin-containing animal material for treating thrombocytopenia, comprising:
(a) hydrolyzing keratin-containing animal materials to obtain a hydrolysate;
(b) filtering and concentrating said hydrolysate obtained in (a);
(c) admixing alcohol; and
(d) purifying.

2. The method of claim 1, wherein said keratin-containing animal materials are animal nails.

3. The method of claim 2, wherein said animal nails are pig nails.

4. The method of claim 1, wherein said hydrolyzing in (a) is performed under acidic, alkaline, or enzymatic conditions.

5. The method of claim 1, wherein said hydrolyzing in (a) is performed at a temperature of between 30 and 150° C.

6. The method of claim 1, wherein said alcohol in (c) is 70% ethanol.

7. The method of claim 1, wherein said purifying in (d) comprises
(i) concentrating solution obtained in (c);
(ii) adjusting the pH to between about 6.5 and 7.5;
(iii) sedimenting; and
(iv) filtering through at most 2.0 μm fibrous membrane

8. The method of claim 1, wherein said purifying in (d) further comprises
(v) adjusting the pH to between about 8 and 10;
(vi) steam heating;
(viii) sedimenting; and
(ix) filtering through at most 2.0 μm fibrous membrane.

9. The method of claim 1, wherein said purifying in (d) further comprises
(v) adjusting pH to between about 2 and 4;
(vi) steam heating;
(viii) sedimenting; and
(ix) filtering through at most 2.0 μm fibrous membrane.

10. The method of claim 1, wherein said purifying in (d) comprises filtering through at most an 80 kDa molecular size membrane.

11. A pharmaceutical composition for treating thrombocytopenia prepared by the method of claim 1.

12. The pharmaceutical composition of claim 11 wherein the molecular weight of proteins present in the pharmaceutical composition is not more than about 80 kDa

13. A pharmaceutical composition for treating thrombocytopenia prepared by the method of claim 3.

14. A pharmaceutical composition for treating thrombocytopenia prepared by the method of claim 7.

15. A pharmaceutical composition for treating thrombocytopenia prepared by the method of claim 8.

16. A pharmaceutical composition for treating thrombocytopenia prepared by the method of claim 10.

17. A method of treating thrombocytopenia comprising administering to a patient in the need thereof the pharmaceutical composition of claim 11.

18. The method of claim 17, wherein said pharmaceutical composition is administered parenterally.

19. The method of claim 18, wherein said pharmaceutical composition is administered intravenously.

20. The method of claim 18, wherein said pharmaceutical composition is administered intramuscularly.
